SEQUENCE LISTING (6 sequences)

SEQ ID NO: 1 - DNA, 1018 bp, Artificial Sequence
  Nucleotide sequence of FZD3 gene
gttcccagattgtgatgagccgtatcctcgacttgtggatctgaatttagttggagatccaactgaaggagccccagtggcagtgcagagggactatggtttttggtgtccccgagaattgaaaattgatcctgatcttggttattcatttttgcacgtgcgtgattgttcacctccttgtccaaatatgtattttaggagagaagaactgtcatttgctcgctatttcataggactgatttcaatcatttgcctttctgccacgttgtttacttttttaacttttttgattgatgtcacaagattccgttatcctgaaaggcctattatattttatgcagtctgctatatgatggtatctttaattttcttcattgggtttttgcttgaagaccgagtagcctgcaacgcatctagccctgcgcaatataaagcttctacagtgacacaaggatctcataataaagcctgtaccatgctttttatggtactttattttttcaccatggctggaagtgtatggtgggtaattcttaccatcacatggtttctagcagctgtgccaaagtggggtagtgaagctattgagaagaaagcattgctatttcacgccagtgcatggggcatccctggaactctaactatcatccttttagcgatgaataaaattgaaggtgacaacattagtggcgtgtgttttgttggcctctacgatgttgacgcattgagatactttgttcttgctcccctctgcctatatgtggtagttggggtttcactcttgttagctggcattatatccctaaacagagttcgaattgagattccattagaaaaggagaaccaagataaattagtgaagtttatgatccggattggtgttttcagcattctttatctcgtaccactcttggttgtaattggatgctacttttacgaacaagcctaccgtggcatctgggaaacaacatggatacaagaacgctgcagagaatatcacattccatgcccatatcag

SEQ ID NO: 2 - DNA, 899 bp, Artificial Sequence
  Nucleotide sequence of KRT26 gene
ccacctgttctagcacttcccaactgcgggaagcttcagaaatgtgtccaggacacgaaacgccgttaaaagtggcgcctggctcccgcctacgacagttctcaagcagcttaatgactttcaggtacgagaacgagctggctctgcaccagagcgtggaggccgacaccaacggcctccgcagggtgctggaggagctgaccctcagcgcggcggacctggagacccagtgcgagaccctccgtgaggagctggcgtatctcaaagcaaaccaccaggaggtaaggagcctgagggcggcttcgatcgccgctctctcccgcttcaagcccgctgctccttccacagcccgcgactgtccgccagggtccccggctgtgggtaggcacacggacccctcttccaggaaatgcaagtcctgcaaagtgcgtcagggggaaacgtgagtgtagagatggacgcagccccgggcgtggacctgactcttctgttgaacaacatgagggctgagtatgagcacctggccgaggagaaccgcagagacgcggaggccaggttcaacgagaaggtacttccgccgcggccggcacacaccgcgcgtgttcagggcacggtctcgcgggccactaacgcttctgaaaatcgctcttcagagtgcgctgctgcagcaacagatttccaatgatgtgggggcagccgcagcagccagaaatgagctgcggagctgaaacgcagcctgcaaaccctggaaatagaactgcagtccctccaggctgtggtatgtcgcaaccaccattgaatcgggggtgtgcgtgagtgtgcatgtgtgtgtgagtgtgtgtatgtgtgtgagtgtgtatgtatgtgtgtgctatgtgtgtatatgtatcagtatatgt

SEQ ID NO: 3 - DNA, 22 bp, Artificial Sequence
  Forward primer for amplification of FZD3 gene
gctggaagtgtatggtgggtaa

SEQ ID NO: 4 - DNA, 22 bp, Artificial Sequence
  Reverse primer for amplification of FZD3 gene
tctcaatgcgtcaacatcgtag

SEQ ID NO: 5 - DNA, 18 bp, Artificial Sequence
  Forward primer for amplification of of KRT26 gene
gtacgagaacgagctggc

SEQ ID NO: 6 - DNA, 18 bp, Artificial Sequence
  Reverse primer for amplification of KRT26 gene
cacagcctggagggactg

(19) United States
(12) Patent Application Publication
(10) Pub. No.: US 2022/0186324 A1
(43) Pub. Date: Jun. 16, 2022

(54) MOLECULAR MARKERS RELATED TO MUTATION OF COARSE WOOL RATE AND WOOL FIBER DIAMETER OF LONG-HAIRED RABBIT AND USE THEREOF

(71) Applicants: Shandong Agricultural University, Tai'an City (CN); Mengyin Yida Rabbit Industry Co., Ltd, Linyi City (CN)

(72) Inventors: Xinzhong FAN, Tai'an City (CN); Aiguo YANG, Tai'an City (CN); Xibo QIAO, Tai'an City (CN); Liang SONG, Tai'an City (CN); Xiaoxue LV, Tai'an City (CN); Jiaqing HU, Tai'an City (CN); Yuanfeng YANG, Tai'an City (CN)

(73) Assignees: Shandong Agricultural University, Tai'an City (CN); Mengyin Yida Rabbit Industry Co., Ltd, Linyi City (CN)

(21) Appl. No.: 17/545,865

(22) Filed: Dec. 8, 2021

(30) Foreign Application Priority Data
Dec. 16, 2020 (CN) 202011500609.7

(51) Int. Cl. C12Q 1/6888; C12Q 1/6858

(52) U.S. Cl. CPC C12Q 1/6888; C12Q 2600/124; C12Q 2600/156; C12Q 1/6858

(57) ABSTRACT

The present disclosure relates to a set of molecular markers. The molecular markers include at least one selected from the group consisting of a mutant of a frizzled class receptor 3 gene (FZD3 gene) and a mutant of a keratin 26 gene (KRT26 gene), where the FZD3 gene is as shown in SEQ ID NO: 1, and the KRT26 gene is as shown in SEQ ID NO: 2; the mutant of the FZD3 gene is formed by mutation of a base T at position 41019916 of a FZD3 gene locus to a base C, and the mutant of the KRT26 gene is formed by mutation of a base G at position 41842284 of a KRT26 gene locus to a base A and/or formed by mutation of a base G at position 41842481 of the KRT26 gene locus to a base C.

### CROSS REFERENCE TO RELATED APPLICATION(S)

This patent application claims the benefit and priority of Chinese Patent Application No. 202011500609.7,filed on Dec. 16, 2020, the disclosure of which is incorporated by reference herein in its entirety as part of the present application.

### TECHNICAL FIELD

The present disclosure relates to the technical field of molecular marker breeding of rabbits, in particular to a set of molecular markers related to a mutation of a coarse wool rate and a wool fiber diameter of a long-haired rabbit and use thereof.

### BACKGROUND ART

The long-haired rabbit coat is mainly composed of coarse hair and fine hair, where the fine hair as a main part has desirable physical and chemical properties and spinnahility. The rabbit wool-based fabric has light texture, smooth and soft hand feel, desirable color, and desirable velvet effect and warmth retention, which is similar to cashmere in many aspects. Rabbit wool production is less restricted by resources, and has lower cost than the cashmere. In addition, the rabbit wool is more acid and alkali resistant, which has advantages over cashmere in cleaning and dyeing. With the improvement of wool spinning technology the development of new products, especially the successful development of worsted rabbit wool fabrics, there is a rapidly increasing demand for fine rabbit wool with desirable homogeneity in domestic and oversea markets.

However, there is currently a lack of high-quality and wool-type long-haired rabbit populations in production. Over the years of long-haired rabbit breeding and production, there has been an excessive pursuit of wool yield, while the improvement of fiber quality is neglected, resulting in an overall thickness of rabbit wool fibers, and the fiber has poor homogeneity in diameter and a high coarse wool rate. This seriously affects the textile quality and fabric value.

The textile value of rabbit wool mainly depends on fiber fineness, length and homogeneity. So far, there are few researches on fiber quality breeding of the long-haired rabbits, and conventional breeding methods can hardly take into account both rabbit wool yield and many other quality indicators. Affected by wool raising cycle, measurement level, population size, genetic evaluation technology, the conventional breeding methods have a relatively poor breeding efficiency and selection accuracy. Therefore, it is of great significance to the cultivation of novel breeds for high-quality and fine-type long-haired rabbits by developing and using effective molecular markers to establish a precise breeding technique for the fiber quality of the long-haired rabbits.

There are many genes involved in regulation of hair follicle development in animals. Frizzled (FZD), encoded by a frizzled 3 gene (FZD3 gene), is an important receptor of the Wnt pathway and binds to Wnt ligands to initiate the Wnt signaling pathway (Youguang Lu, 2009). The FZD3 gene is expressed in the epidermis and cell layers outside the hair follicle. The Wnt controls transcription and expression of downstream target genes through an action of FZD receptor family members on the cell membrane, and plays a vital role in morphogenesis of various types of hair follicles.

Keratin 26 gene (KRT26 gene) can encode a protein of containing 468 amino acids. As a special type-I keratin, The KRT26 is specifically expressed in an internal root sheath of hair follicles and mainly regulates growth and development of the entire hair follicles.

The FZD3 gene and the KRT26 gene are closely related to the performance of hair follicles, and gene structures and function variations of the two genes can affect the hair fiber properties of animals. At present, there is no report about effects of the FZD3 gene and the KRT26 gene on coat traits of the long-haired rabbits.

### SUMMARY

To overcome the above shortcomings of the prior art, the present disclosure is to provide a set of molecular markers related to a mutation of a coarse wool rate and a wool fiber diameter of a long-haired rabbit. The molecular markers have a significant effect on the coarse wool rate (P<0.05), and an extremely significant effect on a variation of the wool fiber diameter (P<0.01) of the long-haired rabbit. In actual breeding work, the molecular markers can provide auxiliary means for selection of the long-haired rabbit on homogeneity of the coarse wool rate and the wool fiber diameter.

To achieve the above objective, the present disclosure adopts the following technical solutions:

A set of molecular markers related to a mutation of a coarse wool rate and a wool fiber diameter of a long-haired rabbit include at least one selected from the group consisting of a mutant of a frizzled class receptor 3 gene (FZD3 gene) and a mutant of a keratin 26 gene (KRT26 gene), where the FZD3 gene is as shown in SEQ ID NO: 1, and the KRT26 gene is as shown in SEQ ID NO: 2; the mutant of the FZD3 gene is formed by mutation of a base T at position 41019916 of a FZD3 gene locus to a base C, and the mutant of the KRT26 gene is formed by mutation of a base G at position 41842284 of a KRT26 gene locus to a base A and/or formed by mutation of a base G at position 41842481 of the KRT26 gene locus to a base C.

In the present disclosure, the base T at the position 41019916 of the FZD3 gene locus of the long-haired rabbit is mutated to the base C. Long-haired rabbits with an allele C have a lower coarse wool rate, while long-haired rabbits with an allele T have a higher coarse wool rate. In the actual breeding, a gene frequency of the allele C in the long-haired rabbit population can be increased to improve the coarse wool rate trait of the long-haired rabbit.

The base G at the position 41842284 of the KRT26 gene locus of the long-haired rabbit is mutated to the base A. Long-haired rabbits with an allele A have a lower coefficient of variation in coarse wool rate and wool fiber diameter, while long-haired rabbits with an allele G have a higher coefficient of variation of coarse wool rate and wool fiber diameter. In the actual breeding, a gene frequency of the allele A can be increased and a gene frequency of the allele G can be reduced in the long-haired rabbit population, to improve wool production traits such as the coarse wool rate and the variation of wool fiber diameter of the long-haired rabbits.

The base G at the position 41842481 of the KRT26 gene locus of the long-haired rabbit is mutated to the base C. Long-haired rabbits with the allele C have a lower coarse wool rate, while long-haired rabbits with the allele T have a higher coarse wool rate. In the actual breeding, a gene frequency of the allele C can be increased and a gene frequency of the allele G can be reduced in the long-haired rabbit population, to improve the coarse wool rate trait of the long-haired rabbit.

The present disclosure further provides use of the molecular markers in marker-assisted selection of a coarse wool rate trait in the long-haired rabbit.

The present disclosure further provides use of the molecular markers in marker-assisted selection of a wool fiber diameter trait in the long-haired rabbit.

A reagent for detecting the molecular markers is provided, where the reagent includes an amplification primer; and the amplification primer includes primers as shown in SEQ ID NO: 3 and SEQ ID NO: 4 for amplifying the FZD3 gene, and/or primers as shown in SEQ ID NO: 5 and SEQ ID NO: 6 for amplifying the KRT26 gene.

Preferably, the reagent for polymerase chain reaction (PCR) may include deionized water, a Taq enzyme, a 10×PCR Buffer and MgCl₂.

The present disclosure further provides a screening method of the molecular markers, including the following steps: constructing a genomic DNA pooling; conducting PCR amplification on a genomic. DNA of an individual sample of a long-haired rabbit to be tested using the primers to obtain PCR products, selecting a gene single nucleotide polymorphism (SNP) site based on the PCR products, and conducting SNP typing using a flight mass spectrometry method to determine whether a base at position 41019916 of a FZD3 gene locus is a base T or mutated to a base C, whether a base at position 41842284 of a KRT26 gene locus is a base G or mutated to a base A. and whether a base at position 41842481 of the KRT26 gene locus is a base G or mutated to a base C.

Preferably, the SNP typing using a flight mass spectrometry method may specifically include the following steps:

S1, according to SNP site information, designing PCR reaction and single-base amplification primers, and conducting quality testing on genomic DNA samples to obtain qualified genomic DNA samples:

S2,subjecting the qualified genomic DNA samples to PCR reaction, and conducting SAP digestion and extension to obtain a reaction product, and

S3, diluting the reaction product, desalting by a resin, spotting a desalted sample on a sample target, crystallizing naturally, and conducting mass spectrometry detection to collect data

Reagents used in the PCR reaction include deionized water, MgCl₂, a PCRBuffer, a dNTPMix and a HotStarTaq.

Preferably, a PCR product amplified by the FZD3 gene may have a length of 750 bp, and a PCR product amplified by the KRT26 gene may have a length of 500 bp.

In the present disclosure, the SNP typing is conducted on the SNP sites existing in the FZD3 gene and the KRT26 gene using a flight mass spectrometry method. Through sequence comparison, it is found that the base T at the position 41019916 of the FZD3 gene locus is mutated to the base C, the base G at the position 41842284 of the KRT26 gene locus is mutated to the base A, and the base G at the position 41842481 of the KRT26 gene locus is mutated to the base C. Association analysis was conducted on the coarse wool rate and the variation of wool diameter of the long-haired rabbit with the FZD3 gene and the KRT26 gene, where when the base T at the position 41019916 of the FZD3 gene locus is mutated to the base C, three genotypes obtained by the SNP typing are closely related to the coarse wool rate trait of the long-haired rabbit, and a coarse w ool rate of a TT genotype is higher than that of a CC genotype and a CT genotype;

when the base G at the position 41842284 of the KRT26 gene locus is mutated to the base A, three genotypes obtained by the SNP typing are closely related to the coarse wool rate trait and the variation of the wool fiber diameter trait of the long-haired rabbit, and a coefficient of variation of a coarse w ool rate and a wool fiber diameter of a GG genotype is higher than that of an AA genotype and a GA genotype; and

when the base G at the position 41842481 of the KRT26 gene locus is mutated to the base C, three genotypes obtained by the SNP typing are closely related to the coarse wool rate trait of the long-haired rabbit, and a coarse wool rate of a long-haired rabbit with a GG genotype is higher than that of a CC genotype and a GC genotype. The above loci have no significant effect on other wool production traits (P>0.05).

The detection of polymorphic sites of the FZD3 gene and the KRT26 gene not only provides new materials for molecular breeding, but also provides scientific basis and auxiliary means for marker-assisted selection of wool production traits in the long-haired rabbit.

Compared with the prior art, the present disclosure has the following beneficial effects:

The present disclosure provides a set of molecular markers related to a mutation of a coarse wool rate and a wool fiber diameter of a long-haired rabbit. The molecular markers have a significant effect on the coarse wool rate (P<0.05), and an extremely significant effect on a variation of the wool fiber diameter (P<0.01) of the long-haired rabbit. In actual breeding work, the molecular markers can provide auxiliary means for selection of the long-haired rabbit on homogeneity of the coarse wool rate and the wool fiber diameter.

### BRIEF DESCRIPTION OF THE DRAWINGS

FIG. 1 shows a schematic diagram of detection results of agarose gel electrophoresis for a PCR amplified product;

FIG. 2-1 shows a schematic diagram of results of cloning sequencing of a PCR amplified product at position 41019916 of a FZD3 gene locus;

FIG. 2-2 shows a gene sequencing peak diagram of the PCR amplified product at the position 41019916 of the FZD3 gene locus;

FIG. 3-1 shows a schematic diagram of results of cloning sequencing of a PCR amplified product at position 41842284 of a KRT26 gene locus;

FIG. 3-2 shows a gene sequencing peak diagram of the PCR amplified product at the position 41842284 of the KRT26 gene locus;

FIG. 4-1 shows a schematic diagram of results of cloning sequencing of a PCR amplified product at position 41842481 of a KRT26 gene locus; and

FIG. 4-2 shows a gene sequencing peak diagram of the PCR amplified product at the position 41842481 of the KRT26 gene locus.

### DETAILED DESCRIPTION OF THE EMBODIMENTS

To better explain the objectives, technical solutions, and advantages of the present disclosure, the present disclosure will be further explained below with reference to accompanying drawings and specific examples.

### EXAMPLES

1. Collection of samples: in the present disclosure, 1009 long-haired rabbit samples in total jointly bred by Shandong Mengyin Yida Rabbit Industry Co., Ltd. and Shandong Agricultural University; were bred under the same conditions as test materials. On the 283th day after birth, shearing was conducted, and the rabbits were raised for wool growth for 73 d, and a wool yield, a coarse wool rate, a wool fiber diameter, a coarse wool diameter and other indicators of each individual were measured, respectively. Wool samples were cut on a center of a side of the rabbit body using scissors, a piece of ear tissue with a size of a soybean grain was cut using surgical scissors; the ear tissue was put into a 1.5 mL centrifuge tube containing 75% alcohol, and taken back to the laboratory, stored at −20° C. for subsequent genomic DNA extraction.

2. Experimental method: a genomic DNA was extracted from the ear tissue sample by high-salt method, and DNA concentration and quality were detected with a spectrophotometer. The extracted DNA was stored at −20° C.

3. Screening gene SNP sites by DNA pooling sequencing

(1) DNA pooling construction: 100 genomic DNA samples were randomly selected, and each 20 genomic DNA samples were mixed in equal volume into a DNA pooling; and

(2) Candidate gene primer design: according to a rabbit FZD3 gene and a rabbit KRT26 gene on an ensemble database, primers for a 5′-regulatory region, a 3′-regulatory region and an exon coding region of the above genes were designed using Primer 5.0, where the primers were synthesized by Sangon Biotech (Shanghai) Co., Ltd., and a primer information was shown in Table 1. Gradient PCR was conducted according to information of the synthesized primers, the best annealing temperature was found to amplify specific target fragments.

TABLE 1
Primers for amplifying genomic DNA

| Primer | | Length/ | Annealing |
|---|---|---|---|
| name | Sequence (5′-3′) | bp | Tm |
| FZD3 | F GCTGGAAGTGTATGGTGGGTAA | 750 | 54.0 |
| | R TCTCAATGCGTCAACATCGTAG | | |
| KRT26 | F GTACGAGAACGAGCTGGC | 500 | 56.8 |
| | R CACAGCCTGGAGGGACTG | | |

4. PCR amplification:

A PCR amplification system was as shown in Table 2:

TABLE 2
PCR amplification system

| Element | Volume (μL) |
|---|---|
| Taq enzyme mixed solution | 12.5 |
| DNA Template | 1.0 |
| Primer F, 10 μM | 1.0 |
| Primer R. 10 μM | 1.0 |
| ddH₂O | 9.5 |
| Total system | 25.0 |

PCR amplification is conducted by:

pre-denaturation at 95° C. for 10 min;

conducting 35 cycles: denaturation at 95° C. for 45 sec;

annealing at 53° C.;

extension at 72° C. for 30 sec; and

extension at 72° C. for 10 min.

After the reaction was complete, PCR products were stored in a refrigerator at 4° C. for sample loading detection and subsequent test analysis.

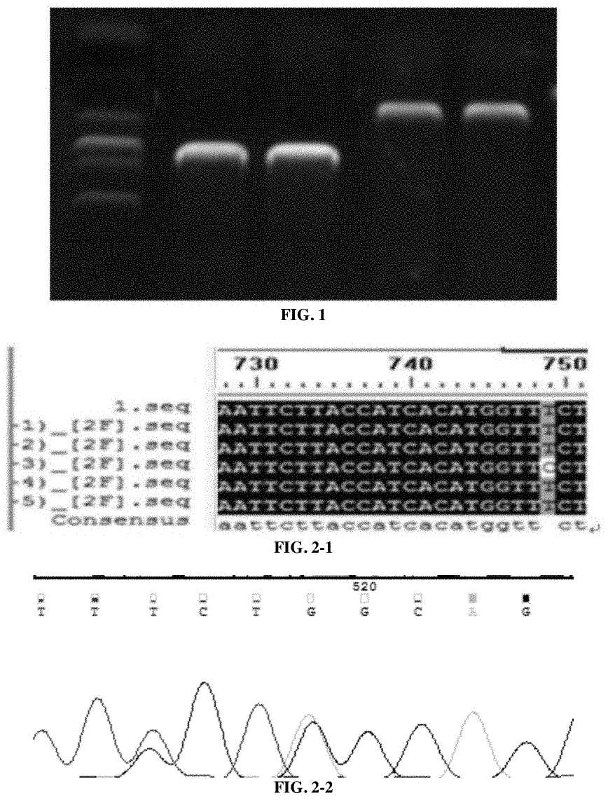

PCR product detection: PCR products obtained were detected using agarose gel electrophoresis. The results are shown in FIG. 1, and it is found that the PCR products have a desirable specificity, and the amplified fragments have the same size as the target fragments, which can be used in the next experiment.

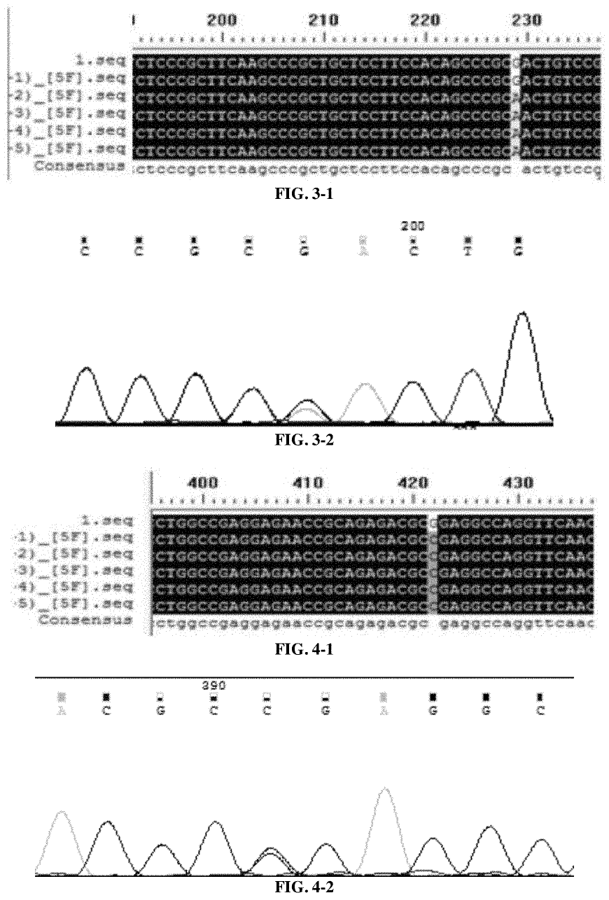

The qualified PCR product was sent to Sangon Biotech (Shanghai) Co., Ltd. for cloning sequencing, and gene SNP sites were selected; SNP typing was conducted by a flight mass spectrometry method; sequencing results were analyzed using a DNAMAN software and a Chromas software to find SNPs. The results are shown in FIGS. 2-1, 2-2, 3-1 and 3-2.

5. SNP typing was conducted by Beijing Compass Biotechnology Co., Ltd., and specific steps were as follows:

(1) Primer design: according to SNP site information, a PCR reaction and single-base extension primers were designed using a software AssayDesignSuitev2.0 designed by MassARRAY, and a specificity of primers was tested online through University of California-Santa Cruz (UCSC) Genome Browser.

(2) Genomic DNA quality inspection: DNA concentration, purity and degree of degradation were detected by agarose gel electrophoresis, where an interpretation standard of test results was: in a gel image of the electrophoresis, the DNA band was single, clear, free of impurities, and there was no dispersion or tailing.

(3) Electrophoresis conditions:

1) 0.8% agarose gel, 170V and 25 min;

2) sample loading volume: 500 ng of a sample +3 μl of a Loading Buffer; and

3) Marker: 3 μl of a Trans2000 Plus.

(4) PCR reaction:

1) A PCR reaction system was shown in Table 3:

TABLE 3
PCR reaction system

| | | |
|---|---|---|
| Reagent | Concentration | Volume (μl) |

| | | |
|---|---|---|
| Water, HPLC grade | NA | 927.5 |
| PCR Buffer with 15 mM MgCl₂ | 10 x | 331.25 |

| | | | |
|---|---|---|---|
| MgCl₂ | 25 | mM | 172.25 |
| dNTP Mix | 25 | mM | 53 |
| Primer Mix | 0.5 | uM | 530 |
| HotStar Taq | 5 | U/μl | 106 |
| DNA template | 10 | ng/ul | 1/well |

| | |
|---|---|
| Total | 5/well |

[symbol]

2) Cycle parameters of the PCR reaction were shown in Table 4:

TABLE 4
PCR reaction cycle parameters

| Temperature (° C.) | Time (second) | Cycle |
|---|---|---|
| 94 | 120 | 1 |
| 94 | 20 | |
| 56 | 30 | 45 |
| 72 | 60 | |
| 72 | 180 | 1 |
| 4 | ∞ | 1 |

(5) SAP digestion

1) A SAP digestion reaction system was shown in Table 5:

TABLE 5
SAP digestion reaction system

| Reagent | Concentration | Volume (ul) |
|---|---|---|
| Water | NA | 810.9 |
| SAP Buffer | 10 x | 90.1 |
| SAP | 1.7 U/ul | 159.0 |
| Total | | 2/well |

2) Cycle parameters of the SAP digestion were shown in Table 6:

TABLE 6
SAP digestion cycle parameters

| Temperature (° C.) | Time (minute) | Cycle |
|---|---|---|
| 37 | 40 | 1 |
| 85 | 5 | 1 |
| 4 | ∞ | 1 |

(6) Extension reaction

1) An extension reaction system was shown in Table 7 below:

TABLE 7
Extension reaction system

| Reagent | Concentration | Volume (ul) |
|---|---|---|
| Water | NA | 400.2 |
| iPLEX buffer plus | 10x | 106 |
| iPLEX terminator | NA | 106 |
| Primer Mix | 0.6-1.3 uM | 426.1 |
| iPlex enzyme | NA | 21.7 |
| total | | 2/well |

2) Cycle parameters of the extension reaction were shown in Table 8 below:

TABLE 8
Extension reaction cycle parameters

| Temperature (° C.) | Time (second) | Cycle |
|---|---|---|
| 94 | 30 | 1 |
| 94 | 5 | 1 |
| 52 | 5 | |
| 80 | 5 | 40 |
| 72 | 180 | 1 |
| 4 | ∞ | |

(7) Detection on machines:

1) the reaction product (9 ul in total) was diluted by 3 times and desalted using a resin;

2) a desalted sample was spotted on a sample target and crystallized naturally; and

3) mass spectrometry detection was conducted to collect data.

According to typing results, population genetic analysis of SNPs and an association analysis of the SNPs with a coarse hair rate trait and a variation of wool fiber diameter trait of the long-haired rabbit were conducted using an R software and a SAS software.

FIGS. 2-1, 3-1 and 4-1 are analyzed, and results show that: through sequence aligntment, it is found that the base T at the 4119916th position of the FZD3 gene locus is mutated to the base C, the base G at the position 41842284 of the KRT26 gene locus is mutated to the base A, and the base G at the position 41842481 of the KRT26 gene locus is mutated to the base C. Meanwhile, different peaks appear on the same sequence site as shown in FIGS. 2-2, 3-2 and 4-2, indicating that base mutations have occurred at this site, where a double peak indicates a heterozygote, and a single peak indicates a homozygote.

Experimental Example Association analysis and population verification on a coarse wool rate trait and a variation of a wool fiber diameter trait of a FZD3 gene and a KRT26 gene of a long-haired rabbit

An analysis of variance was conducted by a general linear model (GLM) procedure using SAS9.2, and an association analysis between each genotype of the polymorphic sites and the wool production trait of the long-haired rabbit was conducted, A best linear unbiased predictor (BLUP) model was:

Y=Xb+Za+e

Y: a phenotypic value of wool yield trait; X: an individual number matrix related to a fixed effect; b: a fixed effect (SNP site, and gender effect); Z: an individual timber matrix of an individual additive genetic effect; a: individual additive genetic effect; and e: a random error.

According to GBT13835 “Rabbit Wool Fiber Test Method”, the coarse wool rate was determined using a test procedure described in a diameter projection microscope method, and a coefficient of variation of the wool fiber diameter was calculated. According to FZD3 gene SNPs and KRT26 gene SNPs, the alleles and genotype frequencies were calculated, and subjected to the Hardy-Weinberg Equilibrium test, to obtain genotypes CC, CT and TT, genotypes AA, AG and GG, and genotypes CC, GC and GG, respectively. The results of each genotype corresponding to the coarse wool rate are shown in Tables 9-11, and the results of the genotypes AA, AG and GG corresponding to the variation coefficient of the wool fiber diameter are shown in Table 10.

TABLE 9
Association analysis and population verification results of
FZD3 gene and coarse wool rate trait of long-haired rabbit

| | Genotype | | | |
|---|---|---|---|---|
| Trait | CC (106) | CT (473) | TT (430) | P value |
| Coarse | 9.32 ± 0.51^{B} | 11.73 ± 0.27^{B} | 12.01 ± 0.35^{A} | <0.01 |
| wool rate | | | | |

TABLE 10
Analysis results of association between mutation of
41842284th position of KRT26 gene and coarse wool
rate and wool fiber diameter of long-haired rabbit

| | Genotype | | | |
|---|---|---|---|---|
| Trait | AA (242) | GA (545) | GG (222) | P value |
| Coarse | 10.55 ± 0.45^{b} | 11.59 ± 0.31^{ab} | 12.50 ± 0.61^{a} | <0.05 |
| wool rate | | | | |
| Coefficient | 14.61 ± 0.24^{B} | 14.73 ± 0.16^{B} | 16.05 ± 0.33^{A} | <0.01 |
| of variation | | | | |
| of wool fiber | | | | |
| diameter | | | | |
| (CVDIA) | | | | |

TABLE 11
Analysis results of association between mutation of 41842481th position
of KRT26 gene and coarse wool rate trait of long-haired rabbit

| | Genotype | | | |
|---|---|---|---|---|
| Trait | CC (217) | GC (564) | GG (228) | P value |
| Coarse | 10.44 ± 0.46^{b} | 11.68 ± 0.30^{a} | 12.54 ± 0.60^{a} | <0.05 |
| wool rate | | | | |

According to the data in Tables 9-11, it can be seen that the position 41019916 of the FZD3 gene locus has a very significant effect on the coarse wool rate of the long-haired rabbit (P<0.01), where a TT genotype is 2.69% and 0.23% higher than that of a CC genotype and a CT genotype, respectively. This base substitution controls 5.18% of the overall genetic variation of the coarse wool rate.

When the base T at the position 41019916 of the FZD3 gene locus is mutated to the base C, the long-haired rabbit with the allele C has a lower coarse wool rate. The long-haired rabbit with the allele T show a higher coarse wool rate. In the breeding work, the coarse wool rate of the long-haired rabbit population can be significantly reduced by establishing a homozygous population of the allele C.

The position 41842284 of the KRT26 gene locus has a significant impact on the coarse wool rate of the long-haired rabbit (P<0.05), and a significant impact on the coefficient of variation of the wool fiber diameter (P<0.01). The GG genotype has a coarse wool rate 1.95% higher than that of the AA genotype; and the GG genotype has a coefficient of variation of wool fiber diameter 1.44% and 1.32% higher than that of the AA genotype and the GA genotype, respectively. This base substitution controls 2.43% of the overall genetic variation of the coarse wool rate and 17.39% of the overall genetic variation of the wool fiber diameter, with an extremely significant genetic effect.

When the base G at the position 41842284 of the KRT26 gene locus is mutated to the base A, the long-haired rabbit with the allele A has lower coarse wool rate and coefficient of variation of the wool fiber diameter; and the long-haired rabbit with the allele G has higher coarse wool rate and coefficient of variation of the wool fiber diameter. In the breeding work, the homogeneity of the coarse wool rate and the wool fiber diameter of the long-haired rabbit can be improved by selecting to increase the gene frequency of the allele A and reduce the gene frequency of the allele G in the long-haired rabbit population.

The position 41842481 of KRT26 gene locus has a significant effect on the coarse wool rate of the long-haired rabbit (P<0.05), where the coarse wool rate of the GG genotype is 2.1% and 0.86% higher than that of the CC genotype and the GC genotype, respectively. When the base G at the position 41842481 of the KRT26 gene locus is mutated to the base C, the long-haired rabbit with the allele C has a lower coarse wool rate, and the long-haired rabbit with the allele G has a higher coarse wool rate. In the breeding work, the coarse wool rate trait of the long-haired rabbit can be improved by increasing the gene frequency of the allele C and reducing the gene frequency of allele G in the long-haired rabbit population.

Finally, it should be noted that the above examples are provided merely to describe the technical solutions of the present disclosure, rather than to limit the protection scope of the present disclosure. Although the present disclosure is described in detail with reference to preferred examples, a person of ordinary skill in the art should understand that modifications or equivalent replacements may be made to the technical solutions of the present disclosure without departing from the spirit and scope of the technical solutions of the present disclosure.

1. A set of molecular markers related to a mutation of a coarse wool rate and a wool fiber diameter of a long-haired rabbit, comprising at least one selected from the group consisting of a mutant of a frizzled class receptor 3 gene (FZD3 gene) and a mutant of a keratin 26 gene (KRT26 gene), wherein the FZD3 gene is as shown in SEQ ID NO: 1, and the KRT26 gene is as shown in SEQ ID NO: 2; the mutant of the FZD3 gene is formed by mutation of a base T at position 41019916 of a FZD3 gene locus to a base C, and the mutant of the KRT26 gene is formed by mutation of a base G at position 41842284 of a KRT26 gene locus to a base A and/or formed by mutation of a base G at position 41842481 of the KRT26 gene locus to a base C.

2. (canceled)

3. (canceled)

4. A reagent for detecting the molecular markers according to claim 1, wherein the reagent comprises an amplification primer; and the amplification primer comprises primers as shown in SEQ ID NO: 3 and SEQ ID NO: 4 for amplifying the FZD3 gene, and/or primers as shown in SEQ ID NO: 5 and SEQ ID NO: 6 for amplifying the KRT26 gene.

5. A screening method of the molecular markers according to claim 1, comprising the following steps: constructing a genomic DNA pooling; conducting polymerase chain reaction (PCR) amplification on a genomic DNA of an individual sample of a long-haired rabbit to be tested using the primers according to claim 4 to obtain PCR products, selecting a gene single nucleotide polymorphism (SNP) site based on the PCR products, and conducting SNP typing using a flight mass spectrometry method to determine whether a base at position 41019916 of a FZD3 gene locus is a base T or mutated to a base C, whether a base at position 41842284 of a KRT26 gene locus is a base G or mutated to a base A, and whether a base at position 41842481 of the KRT26 gene locus is a base G or mutated to a base C.

6. The screening method according to claim 5, wherein the SNP typing using a flight mass spectrometry method specifically comprises the following steps:
S1, according to SNP site information, designing PCR reaction and single-base amplification primers, and conducting quality testing on genomic DNA samples to obtain qualified genomic DNA samples;
S2, subjecting the qualified genomic DNA samples to PCR reaction, and conducting SAP digestion and extension to obtain a reaction product; and
S3, diluting the reaction product, desalting by a resin, spotting a desalted sample on a sample target, crystallizing naturally, and conducting mass spectrometry detection to collect data.

7. The screening method according to claim 5, wherein a PCR product amplified by the FZD3 gene has a length of 750 bp, and a PCR product amplified by the KRT26 gene has a length of 500 bp.
